(19) United States
(12) Patent Application Publication
(10) Pub. No.: US 2024/0398603 A1
(43) Pub. Date: Dec. 5, 2024

(54) Back support brace and method of wearing same

(71) Applicant: Advanced Orthopaedics, Inc., Harleysville, PA (US)

(72) Inventor: Scott Geist, Hilton Head Island, SC (US)

(73) Assignee: Advanced Orthopaedics, Inc., Harleysville, PA (US)

(21) Appl. No.: 18/445,377

(22) Filed: Aug. 4, 2023

Related U.S. Application Data
(60) Provisional application No. 63/577,928, filed on Jun. 2, 2023.

(51) Int. Cl. A61F 5/02

(52) U.S. Cl. CPC A61F 5/02

(57) ABSTRACT

The present invention relates to a back support brace and a method of wearing the same. The back support brace includes a central brace portion formed to rest in abutting support of the back of the person, a pair of adjustable side straps configured to removably attach to the central brace portion on either side thereof and adapted to releasably engage together about the waist of the person, and a means for adjusting the size of the pair of adjustable side straps according to size of the back of the person. In accordance with the present invention, each of the pair of adjustable side straps is contructed and formed having an elasticized insert incorporated into a longitudinal section of the side strap along a lower half thereof to better fit and conform to the transitional area between the waist and hip of the person when the straps are engaged thereby enhancing the comfort of the wearer while reducing unwanted movement of the engaged straps and undesired migration of the back support brace once set in place.

### CROSS-REFERENCE TO RELATED APPLICATION

This application claims the benefit of U.S. Provisional Application Ser. No. 63/577,928 filed Jun. 2, 2023 for a Back Support Brace and Method of Wearing Same.

### FIELD OF THE INVENTION

The present disclosure relates to orthotic devices and in particular, to an improved back support brace construction and method of wearing the same wherein the back support brace includes a set of side straps adapted to a closure system thereby forming a belt, at least one of the side straps being constructed having an elasticized insert incorporated into a longitudinal section of the side strap along a lower half thereof to provide greater comfort to the wearer and prevent migration of the support back support brace once set in place.

### BACKGROUND OF THE INVENTION

Back pain is a very common problem faced by individuals during the course of their lives. Many back issues are treated with medicines or physiotherapy. In addition, back support braces may be worn by individuals to assist in the healing of back injuries. Further, a back support brace promotes good posture, supports back and waist muscles when they are weak, alleviates pain and enables a patient to move around during recovery from any injury.

There are several patents on back support braces or belts that provide back panels or side panels along the belt and belt adjustment means to try and achieve an appropriate fit for the person wearing the same. However, it is extremely difficult to configure a brace with ideal fit without causing discomfort to the person wearing it. Moreover, most of the belts provide adjustment means that wear our very quickly or provide a very clumsy fit around the waist thereby causing discomfort. Those clumsy fitting waist straps can render the entire back support brace ineffective as those straps will move and migrate from their proper postion and cause the back support panels to disengage from their firm position abutting the back and spine.

One such recently granted patent, U.S. Pat. No. 11,324,622 issued Oct. 5, 2022, discloses a prior art back brace that includes a back plate; left and right cord plates each slidably mounted to left and right sides of the back plate, respectively, and to which first and second flexible belt members are respectively secured; and a cord guide arrangement for each plate to particularly. direct first and second cords. While this this prior art back brace is designed to provide adequate support to the wearer, it is quite complex and cumbersome to operate due to its structure having multiple plates, cords with a complex operational arrangement, and a rigid plate structure thereby making it extremely uncomfortable to wear for any extended duration of time. Further, such back brace may not be suitable for patients with certain orthopedic condition that require more flexibility in the back brace to adjust to the structure of the patient's back.

Most of the conventional back support braces are rigid or semi-rigid materials or structures. The problem with a rigid material or structure is that lack of flexibility makes it extremely uncomfortable to wear for long periods and the problem with a semi-rigid or non-rigid material is that the fit is not appropriate and lacks compression to hold the brace in place for longer time thereby making it ineffective.

In view of the above, there is a need for a back support brace which can be worn for extended durations of time without discomfort or migration in its movement, which is easy to operate and fits appropriately to any individual regardless of the waist size.

### SUMMARY OF THE INVENTION

The inventive concepts presented herein are illustrated in a number of different embodiments, each showing one or more concepts, though it should be understood that, in general, the concepts are not mutually exclusive and may be used in combination even when not so illustrated.

Accordingly, in a primary aspect of an inventive embodiment, the subject invention provides an improved back support brace for a person and the associated method for wearing same. The back support brace includes a central brace portion formed to rest in abutting support of the back of the person, a pair of adjustable side straps configured to removably attach to the central brace portion on either side thereof and adapted to releasably engage together about the waist of the person, and a means for adjusting the size of the pair of adjustable side straps according to size of the back of the person. In accordance with the present invention, each of the pair of adjustable side straps is contructed and formed having an elasticized insert incorporated into a longitudinal section of the side strap along a lower half thereof to better fit and conform to the transitional area between the waist and hip of the person when the straps are engaged thereby enhancing the comfort of the wearer while reducing unwanted movement of the engaged straps and undesired migration of the back support brace once set in place.

In a further embodiment, the back support brace of the present invention includes a pair of adjustable pull tabs extending from the central brace portion configured for providing optimal compression to the back when wearing the brace. Further, each of the pair of adjustable pull tabs includes a pull tab size adjusting mechanism configured to enable winding or unwinding of a cord around the size adjusting mechanism for obtaining the optimal compression before locking the pull tab on an exterior portion of the pair of adjustable brace portion.

In another aspect, the pair of adjustable pull tabs may include a pull ring secured to a forward edge of an end sleeve formed having a pocket through which the cord is passed and held secured upon the pull tab size adjusting mechanism removable from the sleeve pocket to adjust the effective cord length by winding or unwinding the cord about the pull tab size adjusting mechanism.

In yet another embodiment, the back support brace may include a central brace portion having means to accommodate an adjustable left side strap and an adjustable right side strap wherein the adjustable left side strap and the adjustable right side strap are configured for adjusting length of the strap depending on the waist and general core size of a person. The back support. brace also includes a removable back pad or shell configured for resting on the central brace portion to provide firm abuting support to the back of the person, and a pair of adjustable pull tab extending from the central brace portion configured for providing optimal compression to the waist when wearing the brace.

In another aspect, the present invention provides a method of wearing a back support brace wherein the method includes the steps of adjusting size of a pair of adjustable side straps according to the size of waist and general core size of a person, attaching the pair of adjustable side straps to a central brace portion by inserting the adjustable side straps inside the central portion, attaching a removable pad or shell on an anterior surface of the central brace portion and placing the removable pad on the back of the person to provide support, placing the pair of adjustable side straps around the waist for closing against each other using a closure system having a plurality of reel and lace mechanism to enable tightening of the brace around the waist, pulling a pair of pull tabs and wrapping a cord upon each of the pull tabs around a notched insert panel for obtaining optimal compression around the waist, and locking the pull tabs on an exterior surface of each of the pair of adjustable side straps for wearing the brace. In the step of placing the adjustable side straps around the waist, each of the pair of adjustable side straps includes an elasticized insert incorporated along a lower half thereof to better fit and conform to the transitional area between the waist and hip of the person thereby maintaining the straps in comfortable position with reduced migration of the back support brace once set in position.

In yet another aspect, the means for adjusting the size of the brace portion includes a reel and lace-based closure system configured for tightening the pair of adjustable side straps around the waist of the wearer by pulling the laces passing through a plurality of eyelets of a pair of plates holding the pair of adjustable side straps.

To further clarify the advantages and features of the present invention, a more particular description of the invention will be rendered by reference to specific embodiments thereof, which is illustrated in the appended figures. It is appreciated that these figures depict only typical embodiments of the invention and are therefore not to be considered limiting of its scope.

### BRIEF DESCRIPTION OF THE DRAWINGS

The above contents of the present invention will become more readily apparent to those ordinarily skilled in the art after reviewing the following detailed description and accompanying drawings, in which:

FIG. 1 presents an exploded view in perspective of a back support brace in accordance with an embodiment of the invention;

FIG. 1A is a plan view of the outer side of the back support brace assembled in accordance with the present invention;

FIG. 1B is a plan view of the inner side of the assembled back support brace with the removable pad or shell resting on the central brace portion of the back support brace in accordance with the present invention;

FIG. 1C is a rear view of the back support brace with its back cover open showing a reel and lace-based closure system employed with an embodiment of the invention;

FIG. 1D shows an adjustable side strap with elasticized insert extending from one side of the central brace portion in accordance with an embodiment of the invention;

FIG. 2 shows a perspective side view of the present back support brace with elasticized insert along the side strap when worn by a person in accordance with an embodiment of the invention; and

FIG. 2A shows a perspective front view of a person wearing the present back support brace with the side straps engaged about the waist while pulling the pull tabs to tighten the brace in accordance with an embodiment of the invention.

Further, skilled artisans will appreciate that elements in the figures are illustrated for simplicity and may not have been necessarily drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION OF THE DRAWINGS

Described herein are nonlimiting example embodiments of the present invention, which includes a back support brace and a method of wearing the same.

The various embodiments including the example embodiments will now be described more fully with reference to the accompanying drawings, in which the various embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the sizes of components may be exaggerated for clarity.

It will be understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “left side strap,” “right side strap,” or “central brace portion,” and the like, may be used herein for ease of description to describe one element or feature's relationship to another element(s) or feature(s) as illustrated in the figures. It will be understood that the spatially relative terms are intended to encompass different orientations of the structure in use or operation in addition to the orientation depicted in the figures.

The subject matter of various embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to a back support brace and a method of wearing the same.

Referring to the drawings, the following is a list of numerical references to elemental parts and components of the present back support brace and those associated structural elements shown throughout the drawings and employed in connection with the present invention:
100 back support brace;
101 central brace portion;
101a removable pad or shell;
102 adjustable left side strap;
103 adjustable right side strap;
104 means to adjust length/size of the pair of adjustable side straps;
105, 106 elasticized inserts;
107, 108 pair of adjustable pull tabs;
109 a reel and lace based closure system;
109a cover flap;
110 laces;
111 plates;
112 eyelets of the plates;
113, 114 end sleeve of the pull tabs;
113a, 114a pockets of the end sleeves;
115, 116 pull tab size adjusting mechanism;
117, 118 rings passing through the end sleeves;
119 cord for wrapping around the pull tab size adjusting mechanism.

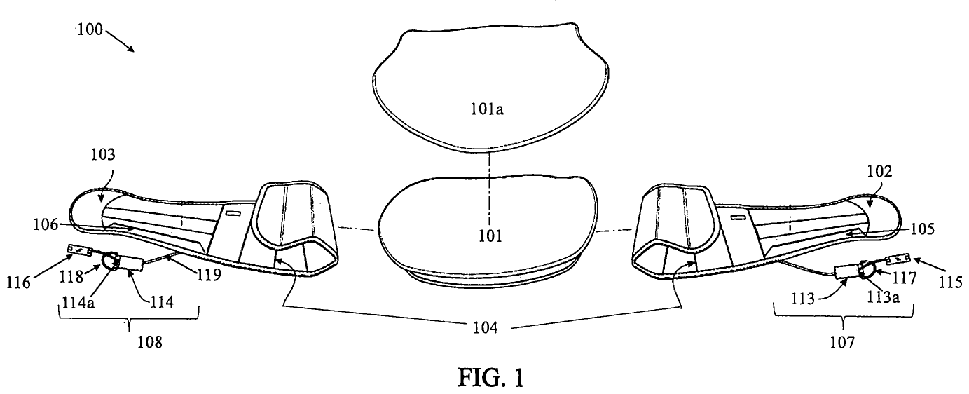

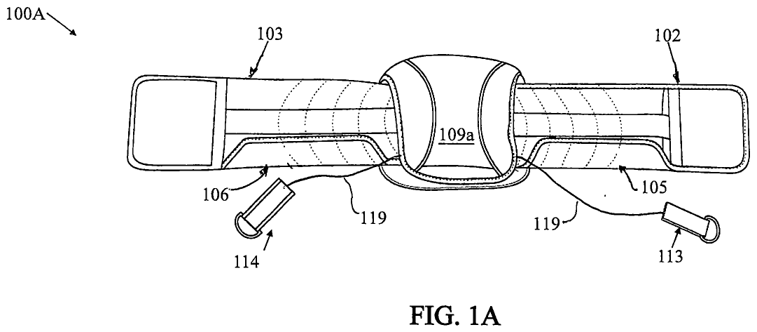

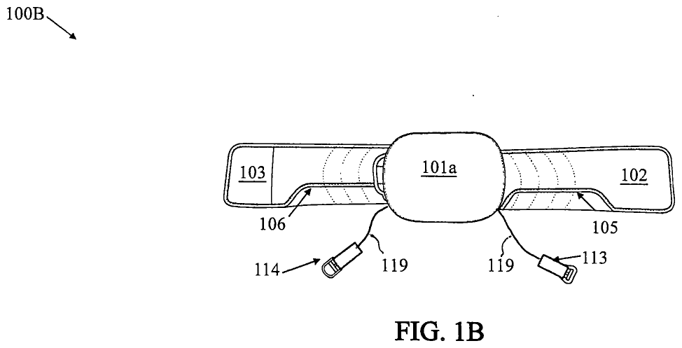

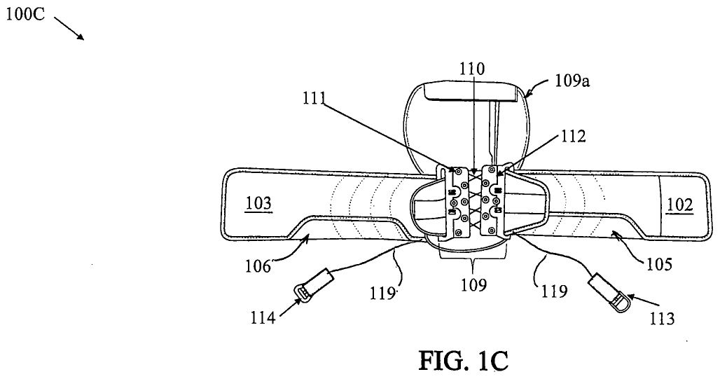

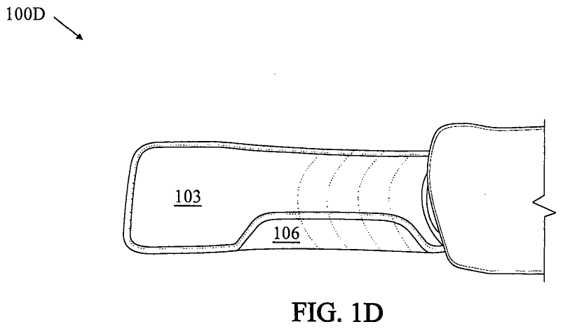

Referring initially to FIG. 1, a preferred embodiment of the present back support brace 100 is shown in a downward perspective of the interior side of the support brace with the major component parts being separated in a partially exploded manner. In accordance with the present invention, the back support brace 100 includes a central brace portion 101, a removable pad or shell 101a, a pair of adjustable side straps 102, 103, each of the side straps having a means 104 to adjust the length thereof and an elasticized insert 105, 106 incorporated into a longitudinal section of the side straps. The back support brace 100 further includes a pair of adjustable pull tabs 107, 108 tethered on either side of the brace and tied via a cord 119 to a reel and lace based closure system 109, better viewed in FIG. 1C, to tighten and comfortably secure the brace around the waist of the wearer.

As viewed in FIGS. 1, 1A and 1B and the back support braces 100, 100A and 100B shown therein, the central brace portion 101 is disposed and formed to rest in support of a back of a person. The central brace portion 101 accommodates the removable back pad or shell 101a positioned to provide firm abutting support to the back of the person when the support brace is tightly secured around the waist of the wearer. The removable pad or shell 101a is releasably fastened to the interior surface of the central brace portion 101 and is the contoured to ensure optimal fit and direct support for the person. The central brace portion 101 is configured on each side to accommodate and engage the respective adjustable side straps 102, 103 so as to form together a belt that may be secured about the waist of the person wearing the back support brace for proper positioned placement of the contoured shell or pad 101a.

In a related aspect of the present embodiment, the pair of adjustable side straps 102, 103 are made to fold in measured sections along their inward lengths proximate to the central brace portion 101. Through engagement with length adjusting means 104 on either side of the central brace portion 101, both the left side strap 102 and right side strap 103 may be folded over and fastened together providing a shorter or longer overall length capable of adjusting the reach of the combined side straps to better fit the waist size of the person wearing the back support brace 100. The pair of adjustable side straps 102, 103 includes one or more foldable sections with the self-fasteners on the interior surface of each that will engage the length adjusting means 104 allowing the sections to fold and refasten so that the side straps may be adjusted in length according to size of the back and waist of the wearer.

Referring to FIG. 1C in conjunction with FIG. 1, the present back support brace 100 further includes a cover flap 109a formed of flexible fabric material fastened along the top of central brace portion 101 with the cover flap being adapted to swing over and upon the back of the central brace portion. In the back view 100C of the support brace 100 of FIG. 1C, the cover flap 109a is opened above the central brace portion 101 to view the reel and lace-based closure system 109 disposed across the back surface of the central brace portion, The reel and lace-based closure system 109 is assembled of extended lengths of a lace material 110 routed through a plurality of eyelets 111 with the lace material being coupled to a pair of plates 112 on either side. The plates 112 are operatively coupled to the side straps 102, 103 on each side of the central brace portion 101 so that the lace material 110 may be drawn and pulled through the eyelets 111 by the plates for tightening the side straps evenly on both sides of the central brace portion and around the waist of the wearer. In a related aspect, the back support brace 100 may further include a pair of side panels positioned on the anterior surfaces of the adjustable side straps 102, 103. These side panels should be planar and smooth in their form and positioned on each of the the side straps 102, 103 near to their rear engagement with the central brace portion 101 on either side thereof to provide firm support to the back of the person.

As viewed in FIGS. 1, 1A, 1B, and 1C and the respective back support braces 100, 100A, 100B and 100C shown therein, the pair of pull tabs 107, 108, Each include an end sleeve 113, 114 with a pocket 113a, 114a formed therein to accommodate a size-adjusting insert mechanism 115, 116 that enables winding or unwinding of the connecting cord 119 before locking the cord in place on the pull tab and fastening the pull tab on an external portion of the adjustable straps 102, 103 to obtain optimal compression of the straps around the waist and hips of the wearer. The pull tabs 107, 108 include a ring 117, 118 passing through the sleeve 113, 114 at one end thereof, where the ring enables the person/wearer to hold and pull the pull tabs to a desired compression state before locking it. The cord 119 is connected to the central brace portion 101 at one end and other end of the cord 109 passes through the end sleeve 113, 114 and connects to the size-adjusting mechanism 115, 116 on each side. In an example embodiment, the size-adjusting mechanism 115, 116 is a notched insert panel having notches on opposite edges that are configured to retain the cord 119 when the cord 119 is wound around and secured within the notch insert panel.

Referring now to FIG. 1D in conjunction with the foregoing FIGS. 1, 1A, 1B and 1C, each of the side straps 102, 103 is constructed predominately of a firm but soft fabric material generally bound about the perimeter thereof to preserve its shape and integrity, further having associated hook and loop fasteners positioned along their respective lengths to provide for their releasable engagement together about the waist and hips of the wearer. Each of the side straps 102, 103 are further constructed with respective elasticized inserts 105, 106 incorporated and secured within an intermediate portion of the lengths of each side strap extending along the lower half thereof in corresponding positions intended to line up with waist and hips of the wearer on both sides. The elasticized inserts 105, 106 thus fitted within the respective side straps 102, 103 beneath the firm strap material thereof will stretch slightly to accommodate and conform to the transitional area between the waist and hip of the person when the straps are engaged while still maintaining the belt-like closure about the wearer. It should be noted and understood that this form and fitted construction of the side straps 102, 103 with the elasticized inserts 105, 106 on each side enhance the comfort of the wearer, reducing unwanted chafing movement of the engaged straps and undesired slipping and migration of the back support brace once set in place.

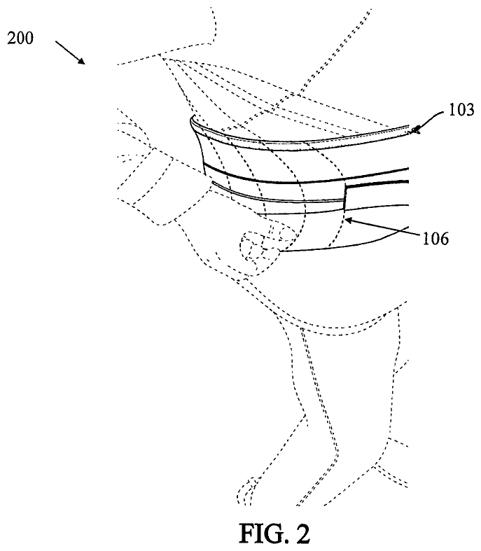

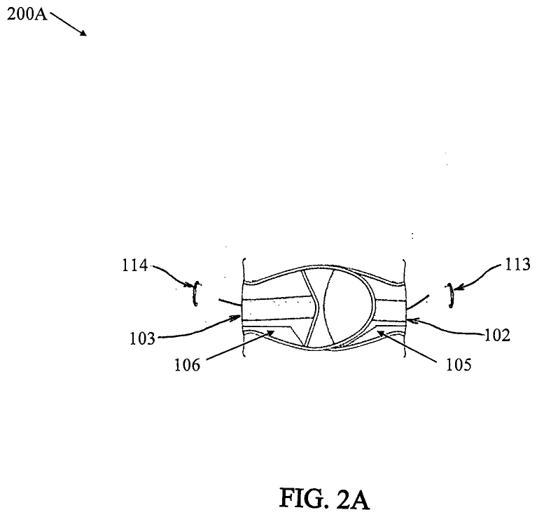

In FIGS. 2 and 2A, the back support brace is shown in respective perpective views 200 and 200A of the brace worn upon a person in accordance with of the present invention. After adjusting the size of the pair of side straps 102, 103 on either said of the central brace portion 101 with the back pad 101a fastened to the interior surface thereof, the side straps are secured around the waist using the tabs 107, 108 on each side to pull on the lengths of cord 119 and in turn, draw on the reel and lace-based closure mechanism 109 that tightens the side straps around the waist with the respective elasticized inserts 105, 106 along the lower half of each strap positioned abutting the waist of the wearer on each side. This closure of the side straps 102, 103 with abutting placement of their respective elasticized inserts 105, 106 conforms securely to the transitional area between the waist and hip of the wearer while maintaining the straps in comfortable position with reduced movement and migration of the back support brace once set in position upon the wearer. In FIG. 2, the right side strap 103 with the elasticized insert 106 running along its lower section is shown in the perspective view 200 of the present back support brace. In FIG. 2A, the left side strap 102 is shown together engaged with the right-side strap 103 with respective elasticized inserts 105, 106 positioned on each side of the wearer. Conventional hook and loop fasteners are employed and positioned on respective surfaces of the left side strap 102 and right side strap 103 to provide for their releasable engagement together across the front of the wearer as well as to the central brace portion 101 at the rear of the back support brace.

In further connection with FIG. 2A, the pair of pull tabs 107, 108 and their respective cords 119 may be adjusted to achieve the required compression. Depending on the waist size of the person, the cords 119 associated with each of the pull tabs 107, 108 are extended and wrapped around the size adjustment mechanism/notched insert panel before inserting back inside the associated covers of the pull tabs to ensure a clean and easy fit. Once the desired compression is achieved, the pull tabs are connected to an exterior surface of the adjustable left side strap 102 and right side strap 103 through the hook and loop fasteners.

While various elements of the back support brace 100 including the side straps 102, 103, central brace portion 101, back pad 101a, back cover flap 109a and pull tabs 107, 108 are shown and described to be releasably connected and assembled using associated hook and loop fasteners, it shall be apparent to a person of ordinary skill in the art that other conventional fasteners may be used for interconnecting and assembling these elements within the scope of this invention.

Variations of the type, form, size, and material of any of central brace portion, adjustable left side strap, right side strap, pull tabs may be of any of those described previously and used in any of the method described.

Obviously, other embodiments and modifications of the present invention will readily come to those of ordinary skill in the art having the benefit of the teachings presented in the foregoing description and drawings. Alternate embodiments of different shapes and sizes, as well as substitution of known materials or those materials which may be developed at a future time to perform the same function as the present described embodiment are therefore considered to be part of the present invention. Furthermore, certain modifications to the described embodiment that serve to benefit its usage are within the scope of the present invention. Accordingly, it is understood that this invention is not limited to the particular embodiment described, but rather is intended to cover modifications within the spirit and scope of the present invention including the appended claims.

What is claimed is:

1. A back support brace for a person wearing same, comprising:
a central brace portion formed to rest in support of a back of the person;
a pair of side straps configured to removably attach to the central brace portion with an extension therefrom on either side thereof to releasably engage together about the waist of the person, each of the pair of side straps being formed having an elasticized insert incorporated into a longitudinal section of the side strap along a lower half thereof to better fit and conform to the transitional area between the waist and hip of the person thereby maintaining the straps in comfortable position with reduced migration of the back support brace once set. in place; and
means for adjusting the extension of the pair of side straps according to size of the back of the person.

2. The back support brace of claim 1, further comprising:
a removable pad or shell disposed on the central brace portion thereby providing support to the back of the person.

3. The back support brace of claim 2, wherein the means for adjusting the extension of the pair of side straps includes a reel and lace-based closure mechanism configured for tightening the pair of side straps around the waist of the wearer by drawing the laces through a plurality of eyelets on a pair of plates holding the pair of side straps on either side of the central brace portion.

4. The back support brace of claim 3, further comprising:
a pair of adjustable pull tab mechanisms extending from the central brace portion on either side for providing optimal compression to the back when wearing the brace.

5. The back support brace of claim 4, wherein each of the pair of adjustable pull tab mechanisms comprise:
a length of cord connected at one end to the central brace portion; and
a notched panel connected to the opposite end of the length of cord and formed to secure the cord wound about the panel to provide optimal compression on an exterior portion of the pair of the side straps.

6. The back support brace of claim 5, wherein each of the pair of adjustable pull tab mechanisms further comprise:
an end sleeve formed having a pocket to hold the notched panel therein with the cord passed therethrough so that the notched panel may be removed from the sleeve pocket to adjust the effective cord length by winding or unwinding the cord about the notched panel; and
a pull ring secured to a forward edge of the end sleeve.

7. The back support brace of claim 1, wherein the pair of side straps includes one or more sections on an anterior surface of each of the pair of adjustable side straps for folding the pair of adjustable side straps to adjust the size according to size of the back of the person.

8. The back support brace of claim 7 wherein the pair of adjustable side straps is attached to the central brace portion through a hook and loop fastener.

9. A back support brace for a person wearing same, comprising:
a central brace portion formed to rest in support of a back of the person;
a pair of adjustable side straps operatively connected to opposite sides of the central brace portion and extending respective lengths therefrom on either side to releasably engage together about the waist of the person, each of the pair of adjustable side straps being formed having an elasticized insert incorporated into a longitudinal section of the side strap along a lower half thereof to better fit and conform to the transitional area between the waist and hip of the person thereby maintaining the straps in comfortable position with reduced migration of the back support brace once set in place; and
means for adjusting the extended lengths of the side straps depending on a waist size of a person.

10. The back support brace of claim 9, wherein each of the adjustable side straps includes one or more sections on an anterior surface of each for folding the side straps to adjust the size according to size of the back of the person.

11. The back support brace of claim 10, wherein the adjustable left side strap and the adjustable right side strap is attached to the central brace portion through hook and loop fastener.

12. A method of wearing a back support brace, comprising:
adjusting size of a pair of adjustable side straps according to the size of waist of a person;
attaching the pair of adjustable side straps to a central brace portion by inserting the adjustable side straps inside the central portion;
attaching a removable pad or shell on an anterior surface of the central brace portion and placing the removable pad on the back of the person to provide support;
placing the pair of adjustable side straps around the waist for locking against each other while tightening of the brace around the waist;
pulling a pair of cord lengths around each side of the side straps and adjusting the cord lengths to obtain optimal compression around the waist; and
locking the adjusted cord lengths on an exterior surface of each of the pair of adjustable side straps for wearing the brace,
wherein the pair of adjustable side straps includes an elasticized insert incorporated into a section of the side straps along a lower half thereof to better fit and conform to the transitional area between the waist and hip of the person thereby maintaining the straps in comfortable position with reduced migration of the back support brace once set in position.

13. The method of claim 12, wherein the pair of adjustable side straps are attached to the central brace portion through hook and loop fasteners.

14. The method of claim 13, wherein the pair of adjustable side straps includes one or more sections on an anterior surface thereof for folding the pair of adjustable side straps to adjust the size according to size of the waist of the wearing person.
